(19) United States
(12) Patent Application Publication
(10) Pub. No.: US 2024/0315948 A1
(43) Pub. Date: Sep. 26, 2024

(54) METHODS FOR ACCLIMATING TO RETINOID-CONTAINING COMPOSITIONS

(71) Applicant: Johnson & Johnson Consumer Inc., Skillman, NJ (US)

(72) Inventors: Xi Li, Shanghai (CN); Hugo Kerriou, Singapore (SG); Yao Sun, Shanghai (CN); Li Ma, Shanghai (CN); Zhang Guodong, Shanghai (CN)

(21) Appl. No.: 18/613,849

(22) Filed: Mar. 22, 2024

Related U.S. Application Data
(60) Provisional application No. 63/454,382, filed on Mar. 24, 2023.

(51) Int. Cl. A61K 8/67; A61Q 19/00

(52) U.S. Cl. CPC A61K 8/671; A61Q 19/00

(57) ABSTRACT

Provided are methods of acclimating skin to a retinoid, the method comprising: topically applying to the skin a first composition comprising a retinoid at a first concentration at a first frequency for a first period of time; topically applying to the skin the first composition at a second frequency for a second period of time; topically applying to the skin a second composition comprising a retinoid at a second concentration at a third frequency for a third period of time; and topically applying to the skin the second composition at a fourth frequency for a fourth period of time.

### CROSS-REFERENCE TO RELATED APPLICATIONS

This application claims the benefit of U.S. Provisional Application 63/454,382 filed on Mar. 24, 2023, the complete disclosure of which is hereby incorporated herein by reference for all purposes.

### FIELD

The present invention generally relates to methods for acclimating skin to retinoid-containing compositions, and particularly methods using compositions comprising retinol.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as hyperpigmentation, acne, greasy or dry skin, keratoses, rosacea, light-sensitive, inflammatory, erythematous, and allergic or autoimmune-reactive reactions, such as dermatosis and photodermatosis.

The consequences of the above-mentioned ageing processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, despite the beneficial effects of retinoid treatment, its benefits are limited due to skin irritation of retinoids. These side effects can restrict the use of retinoids, and particularly so for Asian, and individuals having sensitive skin. Thus, there is an ongoing need to find ways of using retinoids so as to minimize irritation.

### SUMMARY

Accordingly, one aspect of the invention pertains to a method of acclimating skin to a retinoid, the method comprising:
a. topically applying to the skin a first composition comprising a retinoid at a first concentration at a first frequency for a first period of time;
b. topically applying to the skin the first composition at a second frequency for a second period of time;
c. topically applying to the skin a second composition comprising a retinoid at a second concentration at a third frequency for a third period of time; and
d. topically applying to the skin the second composition at a fourth frequency for a fourth period of time.

In one or more embodiments, the first concentration is lower than the second concentration. In some embodiments, the first concentration ranges from about 0.01 to about 1.2 wt. % retinoid by total weight of the composition. In one or more embodiments, the second concentration ranges from about 0.75 to about 2.5 wt. % retinoid by total weight of the composition. In some embodiments, the first frequency is lower than the second frequency. In one or more embodiments, the third frequency is lower than the fourth frequency. In some embodiments, the first frequency is about once every other day. In one or more embodiments, the second frequency is about once every day. In some embodiments, the third frequency is about once every other day or twice per day. In one or more embodiments, the fourth frequency is about once every day or twice per day. In some embodiments, the first, second, and third periods of time are each independently at least from about 5 days to about 12 days. In one or more embodiments, the first, second, and third periods of time are each independently about 7 days. In some embodiments, the fourth period of time is at least about 5 days. In one or more embodiments, the retinoid comprises retinol. In some embodiments, the skin is on the face. In one or more embodiments, the first and second compositions and the first and second concentrations are the same. In some embodiments, the first frequency is about once every third day, the second frequency is about once every other day, and the third frequency is about once every day. In one or more embodiments, the first, second, and third periods of time are each independently about 2 weeks, and the fourth time period is at least one day. In some embodiments, the method further comprises pausing the application of the first or second composition if irritation on the skin occurs. In one or more embodiments, the method further comprises topically applying a moisturizer to the skin until the irritation subsides. In some embodiments, the method further comprises resuming the method at the step previous to the step when paused. In one or more embodiments, the method further comprises topically applying to the skin a composition which is substantially free of a retinoid comprises a moisturizer or skin active agent. In some embodiments, the second time period runs concurrently with the third or fourth time periods.

Another aspect of the invention pertains to a method of acclimating skin to retinol, the method comprising:
a. topically applying to the skin a first composition comprising retinol at a first concentration every other day for a first time period of about 7 days;
b. topically applying to the skin the first composition every day for a second time period of about 7 days;
c. topically applying to the skin a second composition comprising retinol at a second concentration every other day for a third time period of about 7 days; and
d. topically applying to the skin the second composition every day for a fourth time period of at least about 7 days.

In one or more embodiments, the first concentration ranges from about 0.05 to about 1.1 wt. % retinol by total weight of the composition, and the second concentration ranges from about 1.5 to about 2.5 wt. % retinol by total weight of the composition. In some embodiments, the first concentration is about 1 wt. % retinol by total weight of the composition, and the second concentration ranges from about 2 wt. % retinol by total weight of the composition. In one or more embodiments, the skin is on the face. In some embodiments, the fourth time period is at least about 17 weeks. In one or more embodiments, the method further comprises topically applying to the skin a composition which is substantially free of a retinoid comprises a moisturizer or skin active agent.

Another aspect of the invention pertains to a method of acclimating skin to retinol, the method comprising:
a. topically applying to the skin a first composition comprising retinol every third day for a first period of time;
b. topically applying to the skin the first composition every other day for a second period of time; and
c. topically applying to the skin the first composition about once every day for a third period of time.

In one or more embodiments, the first period of time is about 2 weeks. In some embodiments, the second period of time is about 2 weeks. In one or more embodiments, the third period of time is at least about 2 weeks. In some embodiments, the method further comprises pausing the application of the first or second composition if irritation on the skin occurs. In one or more embodiments, the method further comprises topically applying a moisturizer to the skin until the irritation subsides. In some embodiments, the method further comprises resuming the method at the step previous to the step when paused. In one or more embodiments, the method further comprises topically applying to the skin a composition which is substantially free of a retinoid comprises a moisturizer or skin active agent.

### BRIEF DESCRIPTION OF THE DRAWINGS

FIG. 1 is an image of a clinical study participant before and after using a method in accordance with one or more embodiments of the invention;

FIG. 2A-C is a graph showing changes in signs of skin aging of three clinical study groups at 4 weeks, 8 week and 20 weeks;

FIGS. 3A-B show tolerance grading for two clinical study groups over the course of 12 weeks; and

FIGS. 4A-B show the number of adverse events presence for two clinical study groups over the course of 12 weeks.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a composition that is “essentially free” or “substantially free” of an ingredient means the composition that has about 2% or less of that ingredient by weight based on the total weight of the composition. Preferably, a composition that is essentially free of an ingredient has about 1% or less, more preferably about 0.5% or less, more preferably about 0.1% or less, more preferably about 0.05 or less, more preferably about 0.01% or less by weight based on the total weight of composition of the ingredient. In certain more preferred embodiments, a composition that is essentially free of an ingredient is free of the ingredient, i.e. has none of that ingredient in the composition.

As used herein, “cosmetically/dermatologically acceptable” means that the ingredients which the term describes are suitable for use in contact with tissues (e.g., the skin or hair) without undue toxicity, incompatibility, instability, irritation, allergic response, and the like. As will be recognized by one of skill in the art, cosmetically/dermatologically acceptable salts are acidic/anionic or basic/cationic salts.

As used herein, the term “safe and effective amount” means an amount of the compound, extract or of the composition sufficient to induce the desired effect, but low enough to avoid serious side effects. The safe and effective amount of the compound, extract, or composition will vary with e.g. the age, health and environmental exposure of the end user, the duration and nature of the treatment, the specific extract, ingredient, or composition employed, the particular pharmaceutically-acceptable carrier utilized, and like factors.

As used herein, the term “about” refers to within 5% weight, within 4% weight, within 3% weight, within 2.5% weight, within 2% weight, or within 1% weight of a disclosed value.

In general, IUPAC nomenclature rules are used herein and according to the following term definitions.

The term “substituted,” refers to a core molecule in which one or more hydrogen atoms have been replaced with that amount of substituents allowed by available valences. Substitution is not limited to the core molecule, but may also occur on a substituent radical, whereby the radical becomes a linking group.

Acceptable salts from inorganic bases include, for example, sodium or potassium salts, and the like. Acceptable salts from organic bases include, for example, salts formed with primary, secondary, or tertiary amines, and the like.

One or more aspects of the invention pertain to methods of acclimating skin to a retinoid. In one or more embodiments, the method comprises:
a. topically applying to the skin a first composition comprising a retinoid at a first concentration at a first frequency for a first period of time;
b. topically applying to the skin the first composition at a second frequency for a second period of time;
c. topically applying to the skin a second composition comprising a retinoid at a second concentration at a third frequency for a third period of time; and
d. topically applying to the skin the second composition at a fourth frequency for a fourth period of time.

### Retinoid

The retinoid as used herein, refers to a class of compounds that possess the biological activity of Vitamin A in the skin. Examples of retinoids include, but are not limited to, retinol, retinaldehyde, retinoic acid, retinyl palmitate, isotretinoin, tazarotene, bexarotene and adapalene. In certain preferred embodiments, the retinoid is retinol. More preferred are retinol, retinal, or mixtures thereof. Most preferred is retinol. These compounds are well known in the art and are commercially available from a number of sources, e.g., Sigma Chemical Company (St. Louis, Mo.), and Boerhinger Mannheim (Indianapolis, Ind.).

In one embodiment, the retinoid is non-encapsulated. That means the retinoid is not contained in or absorbed onto another material. Advantageously, the retinol is not in the form of the RetiSTAR® commercialized by BASF, since it does not need to be stabilized before being used in the composition according to the present invention.

In general, the retinoid (e.g., retinol) may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, or 10 wt % based on the total weight of the composition. In one or more embodiments, the retinoid (e.g., retinol) may be present in amounts ranging from about 0.001 to about 5 wt %. In further embodiments, the retinoid (e.g., retinol) may be present in amounts ranging from about 0.01 to about 3 wt %. In one or more embodiments, the retinoid (e.g., retinol) may be present in amounts ranging from about 0.01 to about 2 wt %. In further embodiments, the retinoid (e.g., retinol) may be present in amounts ranging from about 0.05 to about 1.5 wt %. In yet further embodiments, the retinoid (e.g., retinol) may be present in amounts ranging from about 0.05 to about 0.5 wt %.

As noted above, there may be more than one concentration that is used in the method. In some embodiments, the first concentration is lower than the second concentration. In one or more embodiments, the first concentration ranges from about 0.01 to about 1.2 wt. % retinoid by total weight of the composition. In some embodiments, the second concentration ranges from about 0.75 to about 2.5 wt. % retinoid by total weight of the composition. In some embodiments, the first concentration is lower than the second concentration. In one or more embodiments, the first concentration ranges from about 0.01 to about 0.5 wt. % retinoid by total weight of the composition.

In some embodiments, the second concentration ranges from about 0.1 to about 2.5 wt. % retinoid by total weight of the composition.

In alternative embodiments, the first and second compositions and the first and second concentrations are the same. That is, one concentration (e.g., from about 0.75 to about 2.5 wt. % or 0.95 to about 1.5 wt. % by total weight of the composition of retinoid) may be used throughout the entire method.

In one or more embodiments, the method further comprises topically applying to the skin a composition which is substantially free of a retinoid comprises a moisturizer or skin active agent.

Such compositions may be used on days when the user following one of the above methods so that the user is still maintaining a routine of applying something to the skin, but still allowing the skin to acclimate by having time off from the retinoid. In one or more embodiments, the composition which is substantially free of a retinoid comprises a moisturizer or skin active agent, or any of the other ingredients discussed above apart from a retinoid (e.g., emollients, humectants, etc.).

### Methods

Another aspect of the invention pertains to a method for treating skin, the method comprising topically applying to skin a composition in accordance with one more embodiments of the invention. As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

Compositions used in the methods of the invention are suitable for improving the texture of skin or improving the firmness of skin, or any of the conditions/symptoms described below.

As used herein, “improving the texture of skin” means the smoothing of the surface of the skin to remove either bumps or crevasses on the skin surface.

As used herein, “improving the firmness of skin” means the enhancing of the firmness or elasticity of the skin, preventing the loss of firmness or elasticity of skin, or preventing or treating sagging, lax and loose skin.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “age spots” means a condition of the skin associated with discrete pigmentation, e.g., small areas of darker pigmentation that may develop on the face as well as the hands.

Signs of skin aging also include the presence of diminished skin thickness, and abnormal or diminished synthesis of collagen, glycosaminoglycans, proteoglycans, elastin, or glycoproteins including fibronectin. In one embodiment, the sign of aging is selected from the abnormal or diminished synthesis of collagen, glycosaminoglycans, proteoglycans, elastin, or glycoproteins including fibronectin. In another embodiment, the sign of skin aging is diminished synthesis of collagen or elastin.

Examples of skin aging that may be treated by topical use of the compositions of this invention include, but are not limited to, wrinkles on the skin. As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

As used herein, “topical use” and “topically applying” means directly laying on or spreading on the skin, hair, or nail, e.g., by use of the hands or an applicator such as a wipe.

The compositions are also suitable for treating or preventing acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that is not visually apparent on the surface of the skin with the naked eye (e.g., as a lesion).

The compositions of the invention are also suitable for treating or preventing rosacea. As used herein, “rosacea” means skin with persistent erythema with or without papules, pustules, or nodules.

The compositions of the invention are also suitable for reducing epidermal hyperkeratinzation. Accordingly, the composition may be used for treatment or prevention of conditions characterized by hyperkeratinzation, such as acne or warts.

In one or more embodiments, one or more of the methods described herein modify the expression of one or more biomarkers. For example, in one or more embodiments, the method may be a method of increasing CRABP2, HAS2, or HBEGF expression in skin. In further embodiments, the method is a method for increasing CRABP2 expression in skin cells, the method comprising topically applying to skin a skincare composition comprising a the retinoid according to the methods described herein.

CRABP2 is well-established in literature as a highly sensitive marker of retinoid bioactivity and potency (Elder J T, Cromie M A, Griffiths C E M, Chambon P, Voorhees J J. Stimulus-selective induction of CRABP-II mRNA: A marker for retinoic acid action in human skin. J Invest Dermatol. 1993; 100(4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18; 11(6):1619-1632). HAS2 is the hyaluronic acid synthase gene that produces hyaluronic acid, which is associated with skin hydration and plumping and is indirectly induced by retinol. Skin plumpness confers antiaging or youthful appearance. Heparin-binding epidermal growth factor (HbEGF). HbEGF is a marker of cell proliferation, which is a feature associated with retinol.

The compositions described herein may be applied to any skin in need of treatment on the human body. For example, application may be made to any one or more of the skin of the face, neck, chest, back, arms, axilla, hands and/or legs. In certain preferred embodiments, the method comprises topically applying a composition according to one or more embodiments of the invention to skin of the face.

Any suitable method of topically applying the composition to the skin in need may be used in accordance with the present invention. For example, the composition may be applied directly from a package to the skin in need, by hand to the skin in need, or may be transferred from a substrate such as a wipe or mask, or a combination of two or more thereof. In other embodiments, the composition may be applied via a dropper, tube, roller, spray, patch or added to a bath or otherwise to water to be applied to the skin, and the like.

In one or more embodiments, the methods of the present invention further comprise the step of leaving the composition in contact with the skin for period of time. For example, in certain preferred embodiments after application, the compound is left in contact with the skin for a period of about 15 minutes or greater. In certain more preferred embodiments, the extract is left in contact with the skin for about 20 minutes or greater, more preferably about 1 hour or greater.

### Frequency

The frequency describes how often the composition is topically applied. The frequency may increase over the span of the method. That is, the first frequency may be lower than the second frequency and/or the third frequency may be lower than the fourth frequency. In some embodiments, the frequency increases while the concentration of retinoid is held the same.

The frequency is generally measured in days. In one or more embodiments, the frequency is twice per day (i.e., every morning and night); or once per day (i.e., every day), every other day, once every third day, once every fourth day, etc. In some embodiments, the first frequency is about once every other day. In one or more embodiments, the second frequency is about once every day. In some embodiments, the third frequency is about once every other day. In one or more embodiments, the fourth frequency is about once every day.

In embodiments where the concentration is maintained, the frequency may change. For example, the first frequency may be about once every third day, the second frequency may be about once every other day, and the third and/or fourth frequency may be about once every day.

### Time Periods

The time periods refer to how long that a given concentration is topically applied at a given frequency. Any time period may span a time period that is at least about 2, 3, 4, 5, 6, 7, 8, 9, 10, 11, 12, 13, 14, 15, or 16 days to about 12, 13, 14, 15, 16, 17, 18, 19, 20 or 21 days (i.e., is at least the number of specified days, but could be longer). In further embodiments, the time period may equal about 2, 3, 4, 5, 6, 7, 8, 9, 10, 11, 12, 13, 14, 15, or 16 days to about 12, 13, 14, 15, 16, 17, 18, 19, 20 or 21 days (i.e., is equal to that length of time). In one or more embodiments, any of the first, second, and third periods of time may be at least from about 5 days to about 12 days. In further embodiments, the first, second, and third periods of time are each independently at least about 7 days, or is equal to about 7 days. In some embodiments, the fourth period of time is at least about 5 days. In one or more embodiments, the first, second, and third periods of time are each independently about 14 days, and the fourth time period is at least one day.

In one or more embodiments, the time periods may overlap with one another. In such situations, the time periods are said to run concurrently. For example, a user may continue to topically apply the first composition comprising a retinoid even after the third and/or fourth time periods have started. In such example, the user may be applying two compositions containing a retinoid in a day.

### Irritation

The methods described herein may further comprise additional steps in the event that irritation of the skin occurs. Such irritation may include, for example, itching, burning, oedema, comedones, pauplae, pustules, erythema, dryness, and scaling. Thus, in one or more embodiments, the method further comprises pausing the application of the first or second composition if irritation on the skin occurs. That is, the user of the regimen may stop progression of the method in order to allow the skin to recover from said irritation. In order to help the skin recover, the user may topically apply a moisturizer to the skin until the irritation subsides. It is thought that applying such a moisturizer may help improve skin barrier function. Once the skin has recovered (e.g., signs of irritation have subsided), then the user may resume the method at the step previous to the step when paused. So, for example, if the user experienced irritation while topically applying the composition once every day, then the user may return to using once every other day once resuming application of the method before proceeding to increase the retinoid concentration and/or frequency.

### EXEMPLARY EMBODIMENTS

The embodiments described herein may be combined in any suitable combination. For example, an exemplary embodiment pertains to a method of acclimating skin to retinol, the method comprising:
a. topically applying to the skin a first composition comprising retinol at a first concentration every other day for a first time period of about 7 days;
b. topically applying to the skin the first composition every day for a second time period of about 7 days;
c. topically applying to the skin a second composition comprising retinol at a second concentration every other day for a third time period of about 7 days; and
d. topically applying to the skin the second composition every day for a fourth time period of at least about 7 days.

In one or more embodiments, the first concentration ranges from about 0.05 to about 1.1 wt. % retinol by total weight of the composition, and the second concentration ranges from about 1.5 to about 2.5 wt. % retinol by total weight of the composition. In further embodiments, the first concentration is about 1 wt. % retinol by total weight of the composition, and the second concentration ranges from about 2 wt. % retinol by total weight of the composition. In some embodiments, the skin is on the face. In one or more embodiments, the fourth time period is at least about 17 weeks.

In another exemplary embodiment, the concentration is maintained over the method. Accordingly, such embodiments pertain to a method of acclimating skin to retinol, the method comprising:
a. topically applying to the skin a first composition comprising retinol every third day for a first period of time;
b. topically applying to the skin the first composition every other day for a second period of time; and
c. topically applying to the skin the first composition about once every day for a third period of time.

In one or more embodiments, the first period of time is about 2 weeks. In some embodiments, the second period of time is about 2 weeks. In one or more embodiments, the third period of time is at least about 2 weeks.

Either of the above exemplary embodiments may also further comprise pausing the application of the first or second composition if irritation on the skin occurs. In further embodiments, the methods further comprise applying a moisturizer to the skin until the irritation subsides. In one or more embodiments, the methods further comprise resuming the method at the step previous to the step when paused.

### Format and Other Ingredients in the Compositions

Any suitable carrier may be used in the compositions of the present invention. Preferably, for a skin care composition, the carrier is a cosmetically-acceptable carrier. As will be recognized by those of skill in the art, cosmetically-acceptable carriers comprise carriers that are suitable for use in contact with the body, in particular the skin for antiaging applications, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 99.8% to about 98% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cP to about 200,000 cP. Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, makeup, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like. In one or more embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The following are non-limitative examples of such carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentylene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99%.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid or dissolvable substrate (eg., a wipe, mask, pad, glove or strip).

The compositions of the present invention may further comprise any of a variety of additional cosmetically active agents, although they are preferably formulated to account for use on skin (skin active agents). Examples of suitable additional active agents include: additional skin lightening agents, darkening agents, anti-acne agents, shine control agents, antimicrobial agents such as anti-yeast agents, anti-fungal, and anti-bacterial agents, anti-inflammatory agents, anti-parasite agents, external analgesics, sunscreens, photo-protectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include hydroxy acids, benzoyl peroxide, D-panthenol, UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH₂, and other botanical extracts such as oat, aloe vera, Feverfew, Soy, Shiitake mushroom extracts, peptides, ceramides, and derivatives and mixtures thereof.

In one or more embodiments, the compositions of the present invention are skin care compositions that further comprise a at least one skin lightening active agent. Examples of suitable skin lightening active agents include, but are not limited to, tyrosinase inhibitors, melanin-inhibiting agents, melanosome transfer inhibiting agents including PAR-2 antagonists, exfoliants, sunscreens, retinoids, antioxidants, Tranexamic acid, skin bleaching agents, allantoin, opacifiers, talcs and silicas, zinc salts, and the like, and other agents as described in Solano et al. Pigment Cell Res. 2006, 19 (550-571). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea coposita root extract, Saxifraga extract and the like, Ellagic acid, Salicylates and derivatives, Glucosamine and derivatives, Fullerene, Hinokitiol, Dioic acid, Acetyl glucosamine, Magnolignane, combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol, phenylethyl resorcinol, 1-(2,4-dihydroxyphenyl)-3-(2,4-dimethoxy-3-methylphenyl)-Propane and the like and natural extracts enriched in resorcinols. Examples of salicylates include, but are not limited to, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, feverfew, parthenolide-free feverfew, oat extracts, pomelo extract, wheat germ extract, Hesperidin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

A variety of other materials may also be present in the compositions of the present invention. These include, for example, chelating agents, humectants, opacifiers, conditioners, preservatives, fragrances and the like. The compositions may include surfactants, for example, those selected from the group consisting of anionic, non-ionics, amphoteric, cationic, or a combination of two or more thereof.

The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

In one or more embodiments, the present invention comprises applying a compound or composition of the invention via a substrate comprising such material. Any suitable substrate may be used in the present invention. Examples of suitable substrates and substrate materials are disclosed, for example, in U.S. Published Application Nos. 2005/022683 and 2009/0241242 which are incorporated herein by reference in their entirety. In certain preferred embodiments, the substrate is a wipe or a facial mask.

The composition and products containing such compositions of this invention may be prepared using methodology that is well known by an artisan of ordinary skill.

### Examples

### Example 1

A clinical study was run testing with retinol tolerance regimen.

### Objectives:

To evaluate the tolerance and efficacy of a skincare regimen on wrinkles and skin sagging
To develop a retinol tolerance-building procedure, particularly for Asian consumers

### Methods:

The study featured a single-center, randomized, controlled, evaluator-blinded, full-face, 20-week clinical trial of participants aged 35-65 years. Evaluations occurred at baseline and 1, 4, 8, 14, and 20 weeks. The assessments included:
Clinical grading on tolerance and efficacy endpoints
Corneometer, Cutometer, AEVA-HE, Visia-CR imaging
Self-assessment questionnaire

The study participants were advised that if irritation occurred, such as scaling, erythema and edema, they were to:
Stop using the product immediately, use thick moisturizers to help strengthen skin barrier
When all symptoms disappear, start using from a less frequent dosage
If symptoms got worse or persisted after discontinuation, they were recommended to consult with a health care professional.

Overview of Study:

| Group 1: Second | Group 2: | Group 3: |
|---|---|---|
| Concentration Only | Regimen | Control |
| (n = 43) | (n = 41) | (n = 24) |
| Second concentration | Face and eye creams at first | Placebo face |
| composition once | concentrations: | cream only |
| daily at night | Once every other day (face | |
| | cream) and night (eye cream) | |
| | at Week 1 | |
| | Once every day (face | |
| | cream) and night (eye cream) | |
| | starting at Week 2 | |
| | Second concentration | |
| | composition added | |
| | Once every day (face | |
| | cream) and night (eye cream), | |
| | once every other night (2^{nd} | |
| | concentration) at Week 3 | |
| | Once every day (face | |
| | cream) and night (eye cream | |
| | and 2^{nd} concentration) | |
| | starting at Week 4 | |

Regular Moisturizer, Sunscreen and Sun protection were recommended during the study.

| | | Eye Cream | Face Cream | |
|---|---|---|---|---|
| | Second | at first | at first | Face |
| | Concen- | concen- | concen- | Cream |
| Composition | tration | tration | tration | Placebo |
| Retinol (w/w %) | 0.10 | 0.02 | 0.05 | 0.00 |

### Results

The table below shows the efficacy endpoint results. All efficacy endpoints in Group land Group 2 were significantly improved compared with baseline from Week 8.

| | | Group 1 - Essence | | | | Group 2 - Regimen | | | | Group 3 - Control | | | |
|---|---|---|---|---|---|---|---|---|---|---|---|---|---|
| Efficacy | Parameters | W 1 | W 4 | W 8 | W 20 | W 1 | W 4 | W 8 | W 20 | W 1 | W 4 | W 8 | W 20 |
| Overall | Overall | — | ✓ | — | ✓ | ✓ | ✓ | ✓# | ✓# | — | ✓ | — | — |
| texture | smoothness | | | | | | | | | | | | |
| & Skin | Overall | — | ✓ | ✓ | ✓ | — | ✓ | ✓ | ✓# | — | — | — | — |
| tone | radiance/ | | | | | | | | | | | | |
| | dullness | | | | | | | | | | | | |
| | Overall | — | ✓ | ✓# | ✓# | — | — | ✓# | ✓# | — | — | — | — |
| | elasticity | | | | | | | | | | | | |
| Fine | Fine lines on | — | ✓ | ✓# | ✓# | — | ✓ | ✓# | ✓# | — | — | — | — |
| lines & | the cheek | | | | | | | | | | | | |
| wrinkles | Fine lines | — | ✓ | ✓# | ✓# | — | ✓# | ✓# | ✓# | — | — | — | — |
| | around eyes | | | | | | | | | | | | |
| | Wrinkles on | — | — | ✓ | #— | — | — | ✓ | ✓# | — | — | — | — |
| | forehead | | | | | | | | | | | | |
| | Glabellar | — | — | — | ✓# | — | — | ✓ | ✓# | — | — | — | — |
| | wrinkles | | | | | | | | | | | | |
| | Crow's feet | — | ✓ | ✓# | ✓# | — | ✓ | ✓# | ✓# | — | — | — | — |
| | Wrinkles on | — | ✓ | ✓# | ✓# | — | — | ✓ | ✓# | — | — | — | — |
| | nasolabial | | | | | | | | | | | | |
| | fold | | | | | | | | | | | | |
| | Wrinkles on | — | — | — | — | — | — | ✓ | ✓# | — | — | — | — |
| | neck | | | | | | | | | | | | |
| Sagging | Eye sagging | — | — | — | ✓ | — | — | — | ✓# | — | — | — | — |
| | Upper cheek | — | — | ✓# | ✓ | — | — | ✓# | ✓# | — | — | — | — |
| | sagging | | | | | | | | | | | | |
| | Jawline | — | — | ✓# | ✓ | — | — | ✓# | ✓# | — | — | — | — |
| | sagging | | | | | | | | | | | | |
| Participants with | | — | 88.37 | 100 | 100 | — | 92.68 | 100 | 100 | — | — | — | — |
| improved pre- | | | | | | | | | | | | | |
| aging/aging signs, % | | | | | | | | | | | | | |
| Hydration | Skin | ✓ | ✓ | ✓ | ✓ | ✓ | ✓ | ✓ | ✓ | — | ✓ | ✓ | ✓ |
| | Hydration | | | | | | | | | | | | |
| Elasticity | Skin elasticity | NA | — | ✓ | ✓ | NA | ✓ | ✓ | ✓ | NA | — | ✓ | ✓ |
| | R2: viscoelasticity | | | | | | | | | | | | |
| | Skin elasticity | NA | ✓ | ✓ | ✓ | NA | ✓ | ✓ | ✓ | NA | — | ✓ | ✓ |
| | R: firmness | | | | | | | | | | | | |
| | Skin elasticity | NA | — | ✓ | ✓ | NA | ✓ | ✓ | ✓ | NA | — | — | ✓ |
| | Q1: elastic | | | | | | | | | | | | |
| | recovery | | | | | | | | | | | | |

✓ = significant improvement compared with baseline, P < 0.05
# = significant improvement compared with control, P < 0.05
— = no significant improvement compared with both baseline and control,
N/A = not measured at this visit

As seen from the above table, Group 2 experienced significant improvement in all categories related to texture, skin tone, fine lines, wrinkles and sagging compared with the baseline and control. In comparison, Group 1 experienced significant improvement with respect to baseline only for several of these categories.

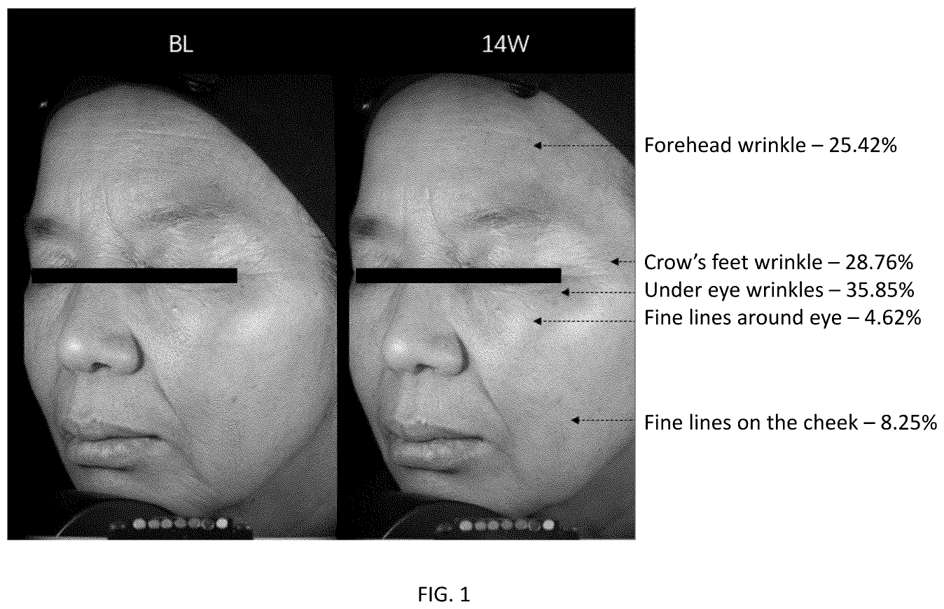

FIG. 1 shows an ALVA-HE 4D image of one of the study participants. As can be seen from the figure, there is significant reduction in forehead wrinkles, crow's feet wrinkles, under eye wrinkles, fine lines around the eye and fine lines on the cheek.

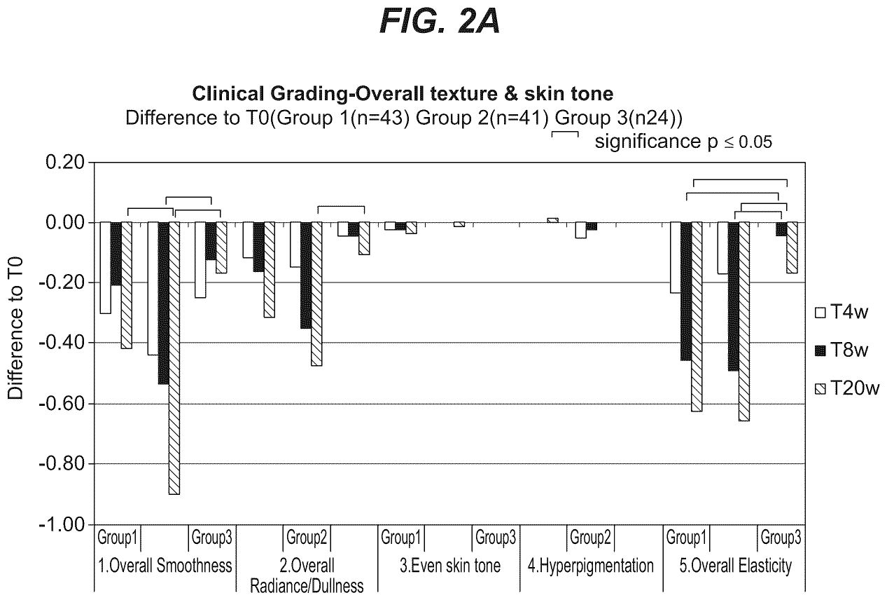

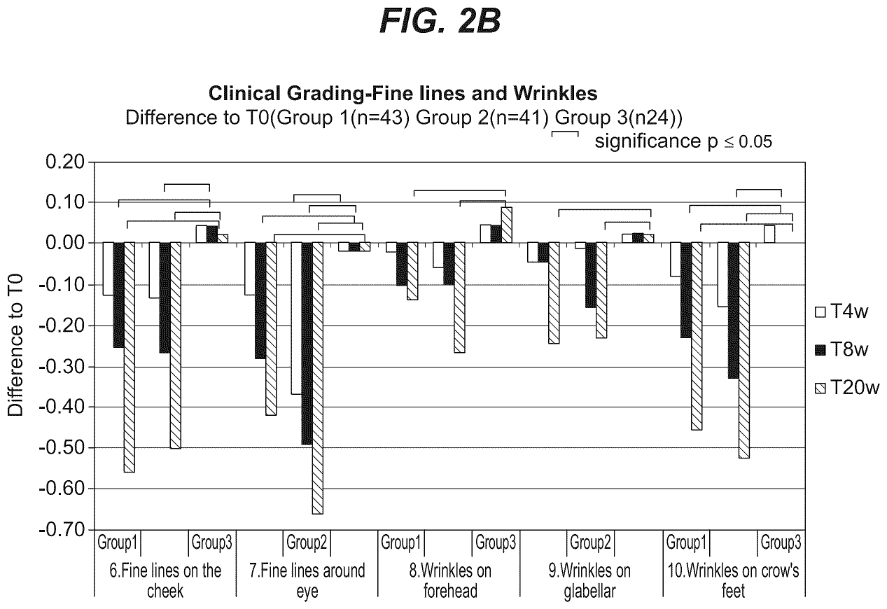

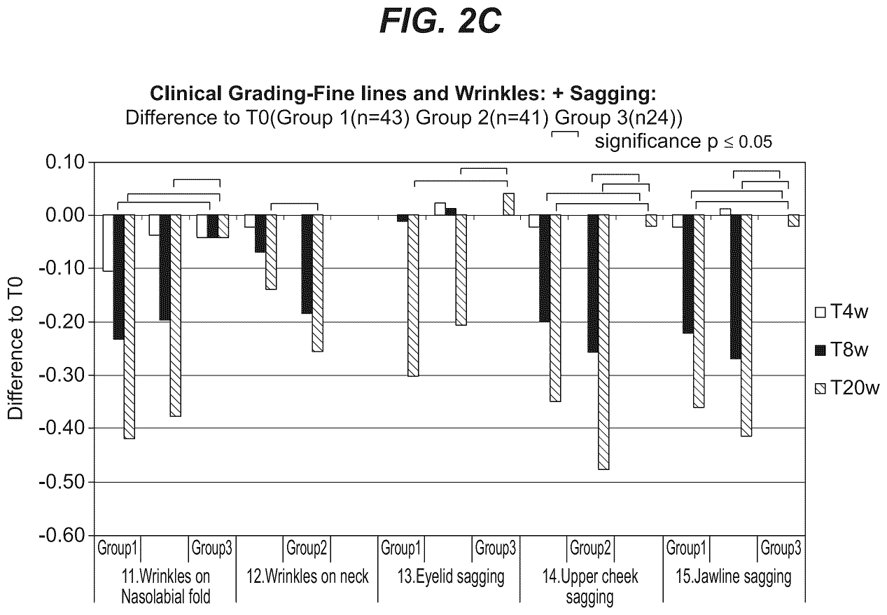

FIGS. 2A-C show clinical grading results of the study. When compared among groups:

Significant difference was documented at T4w between Group 2 and 3 for “Fine lines around eye”, in favour of Group 2.

Significant difference was documented at T8w between Group 1 and 3 for “Overall Elasticity”, “Fine Lines on the cheek”, “Fine lines around eye”, “Wrinkles on crow's feet”, “Wrinkles on Nasolabial fold”, “Upper cheek sagging” and “Jawline sagging”, in favour of Group 1; significant difference was documented at T8w between Group 2 and 3 for “Overall Smoothness”, “Overall Elasticity”, “Fine Lines on the cheek”, “Fine lines around eye”, “Wrinkles on crow's feet”, “Upper cheek sagging” and “Jawline sagging”, in favor of Group 2.

Significant difference was documented at T20w between Group 1 and 2 for “Overall Smoothness”, in favor of Group 2; between Group 1 and 3 for all parameters except “Overall Smoothness”, “Overall Radiance/Dullness”, “Even skin tone”, “Hyperpigmentation” and “Wrinkles on neck”, in favor of Group 1; Between Group 2 and 3 for all parameters except “Even skin tone” and “Hyperpigmentation”, in favor of Group 2.

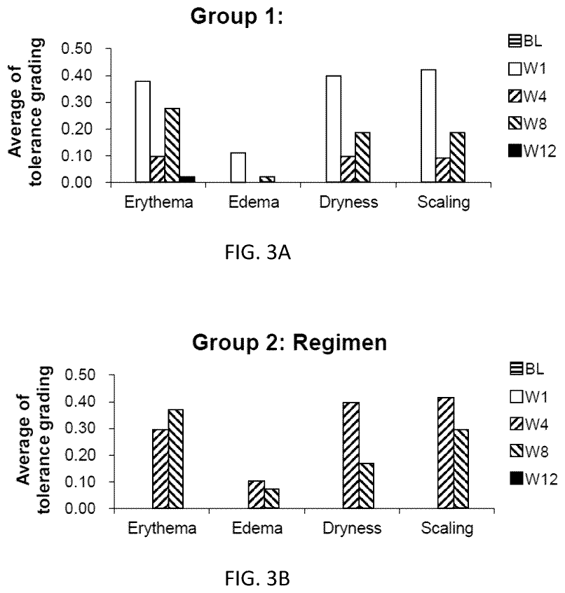

FIG. 3A-B show the erythema, edema, dryness and scaling across 12 weeks for Group 1 and Group 2, respectively. The control group did not experience any issues with tolerance as they did not receive any retinol. Retinol-related irritation, including erythema, dryness, and scaling, occurred mainly during the first month. As can be seen, although both groups experienced tolerance issues, Group 2 experienced issues with erythema, dryness and scaling over a two week period only, and similar % of subjects build tolerance to a higher dose 3 products regiments eventually; whereas Group 1 experienced these symptoms over a period of 3-4 weeks by using consistently with single lower dose retinol products. Thus, the regimen helps to shorten the period of adjustment.

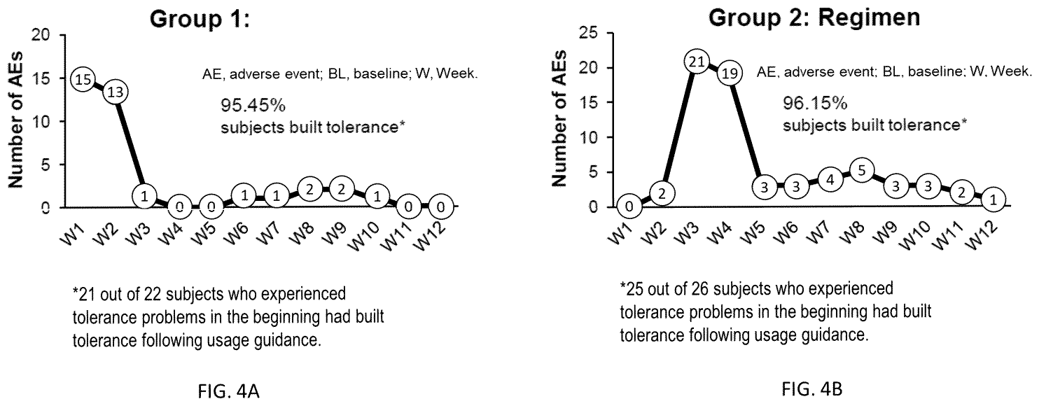

FIGS. 4A-B shows the number of adverse events over the 12 week period. The figures show that both groups 95% and 96% of subjects were tolerant to the irritation for Groups 1 and 2, respectively.

### Prophetic Example 2

A retinol composition is given to a user. The retinol composition may have a typical dosing of retinol (e.g., about 1 wt. % by total weight of the composition). The user is given instructions to use the retinol composition according to the following schedule:

| Weeks 1-2 | Weeks 3-4 | Weeks 5-6 |
|---|---|---|
| Once every third day | Once every other day | Once everyday |

However, if irritation occurs, such as scaling, erythema and edema, the user is given the following instructions;
Stop using the product immediately, use thick moisturizers to help
strengthen skin barrier.
When all symptoms disappear, start using from a less frequent dosage.
If your symptoms get worse or persist after discontinuation, we recommend that you consult with your health care professional.

The table below shows a prophetic example. The user begins the treatment by using the retinol composition on their face once every third day (denoted by “O” in the table below). However, starting with Week 3, the user develops irritation on the skin (denoted by *). The user thus pauses the treatment over the next week. During this time, they use a moisturizer that is though to help with the skin barrier. At this point, the irritations subside. The user then resumes the treatment at the initial frequency (i.e., once every three days). After two weeks of successfully completing the treatment at a frequency of once every three days, the user progresses to the next frequency of once every other day and then move to next level frequency (everyday) once stabilized for 2 weeks until the user completes the treatment.

| | Day | Day | Day | Day | Day | Day | Day |
|---|---|---|---|---|---|---|---|
| | 1 | 2 | 3 | 4 | 5 | 6 | 7 |
| Week 1 | ◯ | — | — | ◯ | — | — | — |
| Week 2 | — | — | ◯ | — | — | ◯ | — |
| Week 3 | * | — | — | — | — | — | — |
| Week 4 | ◯ | — | — | ◯ | — | — | — |
| Week 5 | — | — | ◯ | — | — | ◯ | — |
| Week 6 | ◯ | — | ◯ | — | ◯ | — | ◯ |
| Week 7 | ◯ | — | ◯ | — | ◯ | — | ◯ |
| Week 8 | ◯ | ◯ | ◯ | ◯ | ◯ | ◯ | ◯ |
| Week 9 | ◯ | ◯ | ◯ | ◯ | ◯ | ◯ | ◯ |

What is claimed is:

1. A method of acclimating skin to a retinoid, the method comprising:
a. topically applying to the skin a first composition comprising a retinoid at a first concentration at a first frequency for a first period of time;
b. topically applying to the skin the first composition at a second frequency for a second period of time;
c. topically applying to the skin a second composition comprising a retinoid at a second concentration at a third frequency for a third period of time; and
d. topically applying to the skin the second composition at a fourth frequency for a fourth period of time.

2. The method of claim 1, wherein the first concentration is lower than the second concentration.

3. The method of claim 1, wherein the first concentration ranges from about 0.01 to about 1.2 wt. % retinoid by total weight of the composition.

4. The method of claim 1, wherein the second concentration ranges from about 0.75 to about 2.5 wt. % retinoid by total weight of the composition.

5. The method of claim 1, wherein the first frequency is lower than the second frequency.

6. The method of claim 1, wherein the third frequency is lower than the fourth frequency.

7. The method of claim 1, wherein the first frequency is about once every other day.

8. The method of claim 1, wherein the second frequency is about once every day.

9. The method of claim 1, wherein the third frequency is about once every other day or twice per day.

10. The method of claim 1, wherein the fourth frequency is about once every day or twice per day.

11. The method of claim 1, wherein the first, second, and third periods of time are each independently at least from about 5 days to about 12 days.

12. The method of claim 11, wherein the first, second, and third periods of time are each independently about 7 days.

13. The method of claim 11, wherein the fourth period of time is at least about 5 days.

14. The method of claim 1, wherein the retinoid comprises retinol.

15. The method of claim 1, wherein the skin is on the face.

16. The method of claim 1, wherein the first and second compositions and the first and second concentrations are the same.

17. The method of claim 16, wherein the first frequency is about once every third day, the second frequency is about once every other day, and the third frequency is about once every day.

18. The method of claim 17, wherein the first, second, and third periods of time are each independently about 2 weeks, and the fourth time period is at least one day.

19. The method of claim 1, further comprising pausing the application of the first or second composition if irritation on the skin occurs.

20. The method of claim 19, further comprising topically applying a moisturizer to the skin until the irritation subsides.

21. The method of claim 19, further comprising resuming the method at the step previous to the step when paused.

22. The method of claim 1, further comprising topically applying to the skin a composition which is substantially free of a retinoid comprises a moisturizer or skin active agent.

23. The method of claim 1, wherein the second time period runs concurrently with the third or fourth time periods.
